(19) United States
(12) Patent Application Publication
(10) Pub. No.: US 2010/0159522 A1
(43) Pub. Date: Jun. 24, 2010

(54) ORGANOSOLV AND OZONE TREATMENT OF BIOMASS TO ENHANCE ENZYMATIC SACCHARIFICATION

(75) Inventor: JELENA CIRAKOVIC, Wilmington, DE (US)

Correspondence Address: E I DU PONT DE NEMOURS AND COMPANY;LEGAL PATENT RECORDS CENTER, BARLEY MILL PLAZA 25/1122B, 4417 LANCASTER PIKE, WILMINGTON, DE, 19805, US

(73) Assignee: E.I. DE PONT DE NEMOURS AND COMPANY, Wilmington, DE (US)

(21) Appl. No.: 12/640,351

(22) Filed: Dec. 17, 2009

Related U.S. Application Data
(60) Provisional application No. 61/139,106, filed on Dec. 19, 2008.

(51) Int. Cl. C12P 19/00; C07H 1/08

(52) U.S. Cl. 435/72; 536/128

(57) ABSTRACT

Lignocellulosic biomass comprising lignin is treated with a solvent, such as organosolv, under alkaline conditions at elevated temperatures, filtered, then contacted with a gas comprising ozone to produce a readily saccharifiable biomass.

### CROSS-REFERENCE TO RELATED APPLICATION

This application claims benefit of priority from Provisional Application No. 61/139,106 filed Dec. 19, 2008. This application hereby incorporates by reference Provisional Application No. 61/139,106 in its entirety.

### FIELD OF THE INVENTION

Methods for producing readily saccharifiable, carbohydrate-enriched lignocellulosic biomass are provided and disclosed. Specifically, pretreated biomass is prepared through simultaneous fragmentation and selective extraction of lignin under alkaline organosolv conditions at elevated temperatures, then by contacting with a gas comprising ozone. The remaining carbohydrate-enriched solids in the pretreated biomass may then be subjected to enzymatic saccharification to obtain fermentable sugars, which may be subjected to further processing for the production of other target products.

### BACKGROUND OF THE INVENTION

Cellulosic and lignocellulosic feedstocks and wastes, such as agricultural residues, wood, forestry wastes, sludge from paper manufacture, and municipal and industrial solid wastes, provide a potentially large renewable feedstock for the production of chemicals, plastics, fuels and feeds. Cellulosic and lignocellulosic feedstocks and wastes, composed of carbohydrate polymers comprising cellulose, hemicellulose, pectins and lignin are generally treated by a variety of chemical, mechanical and enzymatic means to release primarily hexose and pentose sugars, which can then be fermented to useful products.

Pretreatment methods are usually used to make the polysaccharides of lignocellulosic biomass more readily accessible to cellulolytic enzymes. One of the major impediments to cellulolytic enzyme digest of polysaccharide is the presence of lignin, a barrier that limits the access of the enzymes to their substrates, and a surface to which the enzymes bind non-productively. Because of the significant cost of enzyme in the pretreatment process, it is desirable to minimize the enzyme loading by either inactivation of the lignin to enzyme adsorption or its outright extraction. Another challenge is the inaccessibility of the cellulose to enzymatic hydrolysis either because of its protection by hemicellulose and lignin or by its crystallinity. Pretreatment methods that attempt to overcome these challenges include: steam explosion, hot water, dilute acid, ammonia fiber explosion, alkaline hydrolysis (including ammonia recycled percolation), oxidative delignification, organosolv, and ozonation.

Published PCT patent application WO 97/36040 discloses a process for the bleaching of organosolv pulp comprising the steps of bleaching with ozone a brownstock pulp obtained from an organosolv pulping process; washing the ozonated pulp with an aqueous solution of a lower alkyl alcohol to remove lignin dissolved therein; and removing filtrates of the aqueous solution from the washed pulp. The process further comprises the step of washing the brownstock pulp with an aqueous solution of a lower alkyl alcohol prior to ozonating the pulp.

J. Quesada et al., in Journal of Applied Polymer Science (1998), 68, 1867-1876, disclose studies of the solubility of lignin from autohydrolyzed corn stalks in different organic solvent-water mixtures and under different conditions, followed by ozonation of both the solvolytic solid and the juice. Optimal conditions for organosolv treatment were 75/25 (v/v) acetone/water mixture at 210° C. for 45 minutes. After organosolvolysis, the material was treated in a fixed bed reactor at room temperature for 20 minutes with ozone. Prior to being used in the studies, the autohydrolyzed corn stalks were prepared as follows: dried at ambient temperature, ground in a hammer mill, sieved to a particle size of 0.2-0.5 mm, soxhlet-extracted with a 7/3 (v/v) ethanol-toluene mixture, soxhlet-extracted with water, submitted to autohydrolysis at 220° C. for 3 minutes in a steam explosion device, extracted with abundant water at ambient temperature, and then extracted with one liter of boiling water.

Previously applied pretreatment methods often suffer from shortcomings, including separate hexose and pentose streams, inadequate lignin extraction or lack of separation of extracted lignin from polysaccharide, particularly in those feedstocks with high lignin content (e.g., sugar cane bagasse, softwoods), disposal of waste products (e.g., salts formed upon neutralization of acid or base), and poor recoveries of carbohydrate due to breakdown or loss in wash steps. Other problems include the high cost of energy, capital equipment, and pretreatment catalyst recovery, and incompatibility with saccharification enzymes.

One of the major challenges of the pretreatment of lignocellulosic biomass is to maximize the extraction or chemical neutralization (with respect to non-productive binding of cellulolytic enzymes) of the lignin while minimizing the loss of carbohydrate (cellulose plus hemicellulose). The more carbohydrate that can be retained, the higher will be the overall yield of monomeric sugars following combined pretreatment and enzymatic saccharification.

A method for selective extraction of lignin from biomass to produce carbohydrate-enriched biomass in a cost effective manner is desired. Also desired is a method for producing carbohydrate-enriched biomass which is highly susceptible to enzymatic saccharification and can produce high yields of fermentable sugars (for example, glucose and xylose) for their bioconversion to value-added chemicals and fuels.

### SUMMARY OF THE INVENTION

The present invention provides methods for producing readily saccharifiable carbohydrate-enriched biomass and for selectively extracting and fragmenting lignin while retaining carbohydrate in good yield. The methods include treating lignocellulosic biomass with a solvent, such as organosolv, under alkaline conditions at elevated temperatures, then contacting the biomass with a gas comprising ozone. Following pretreatment, the biomass may be further treated with a saccharification enzyme consortium to produce fermentable sugars. These sugars may be subjected to further processing for the production of target products. In one embodiment of the invention, a method is provided, the method comprising:

(a) providing lignocellulosic biomass comprising lignin;

(b) contacting the biomass with an organic solvent solution comprising water and at least one inorganic base selected from the group consisting of sodium hydroxide, sodium carbonate, potassium hydroxide, potassium carbonate, calcium hydroxide, calcium carbonate, ammonia, and mixtures thereof whereby a biomass-solvent suspension is formed;

(c) heating the biomass-solvent suspension to a temperature of about 100° C. to about 220° C. and for a reaction time of about 15 minutes to about 48 hours whereby lignin is fragmented from the biomass and is dissolved in the suspension;

(d) filtering free liquid under pressure after heating the suspension in (c) whereby the dissolved lignin is removed whereby pretreated biomass is formed;

(e) contacting the pretreated biomass from (d) with a gas comprising ozone at a temperature of about 0° C. to about 50° C. and for a reaction time of at least about 1 minute whereby a readily saccharifiable carbohydrate-enriched biomass is produced.

In some embodiments, the method further comprises: after (d) and before (e), washing the pretreated biomass produced in step (d) with the organic solvent solution. In some embodiments, the lignocellulosic biomass is subjected to preprocessing prior to step (a), with the proviso that such preprocessing is not required to produce the readily saccharifiable carbohydrate-enriched biomass.

In some embodiments, the temperature in (c) is about 140° C. to about 180° C. In some embodiments, the reaction time in (c) is about 1 hour to about 12 hours.

According to the methods of the invention, the organic solvent solution further comprises an alcohol selected from the group consisting of methanol, ethanol, n-propanol, isopropanol, n-butanol, 2-butanol, isobutanol, and t-butanol, and mixtures of these. In some embodiments, the alcohol is ethanol. In some embodiments, the organic solvent solution contains about 0 to about 100 percent (volume/volume) ethanol. In some embodiments, the amount of the inorganic base is about 1 weight percent to about 14 weight percent, relative to dry weight of biomass. In some embodiments, the inorganic base is potassium carbonate. In some embodiments, the dry weight of biomass is at a concentration of from about 15% to about 70% of the weight of the biomass-solvent suspension of (b).

According to the methods of the invention, the gas comprises about 0.1 to about 20 percent by volume ozone. In some embodiments, the gas comprises about 0.5 to about 5 percent by volume ozone. In some embodiments, the gas further comprises air, nitrogen, oxygen, argon, or a combination thereof. In some embodiments, the ratio of ozone to pretreated biomass in step (e) is at least about 1:1200 on a weight basis. In some embodiments, the temperature in (e) is about 0° C. to about 25° C.

In some embodiments, the method further comprises, prior to step (e), a step of contacting the pretreated biomass with a second solvent solution comprising water, whereby a second biomass-solvent suspension is formed. In some embodiments, the weight percent of biomass in the second biomass-solvent suspension is from about 20 to about 70. In some embodiments, the second solvent solution comprises a buffer. In some embodiments, the second biomass-solvent suspension has a pH of about 1 to about 10. In some embodiments, the method further comprises, after step (e), adjusting the pH to a second pH sufficient for enzymatic saccharification of the biomass. In some embodiments, the second solvent solution further comprises an alcohol selected from the group consisting of methanol, ethanol, n-propanol, isopropanol, n-butanol, 2-butanol, isobutanol, and t-butanol, and mixtures of these. In some embodiments, the alcohol is ethanol. In some embodiments, the second solvent solution contains about 0 to about 100 percent (volume/volume) ethanol.

In some embodiments, the method further comprises applying energy to the pretreated biomass during step (e). In some embodiments, the method further comprises applying energy to the pretreated biomass during step (e), wherein the applying energy is by milling, crushing, grinding, shredding, chopping, disc refining, ultrasound, microwave, or a combination of these. In some embodiments, the pretreated biomass contains about 30 to about 60 percent moisture.

In some embodiments, the methods of the invention further comprise saccharifying the biomass with an enzyme consortium whereby fermentable sugars are produced. In some embodiments, the methods of the invention further comprise fermenting the sugars to produce a target product. In some embodiments, the target product is selected from the group consisting of ethanol, butanol, and 1,3-propanediol.

### DETAILED DESCRIPTION OF THE INVENTION

Applicants specifically incorporate the entire contents of all cited references in this disclosure. Further, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

The present invention provides a two-step method for the treatment of biomass in order to enhance the subsequent enzymatic saccharification step. In the first step, lignin is simultaneously fragmented and extracted using alkaline organosolv conditions at elevated temperatures. In the second step, the biomass, as a solvent suspension or as a solid, is contacted with a gas comprising ozone. The treated biomass may be digested with a saccharification enzyme consortium to produce fermentable sugars.

### DEFINITIONS

The following definitions are used in this disclosure:

“Room temperature” and “ambient” when used in reference to temperature refer to any temperature from about 15° C. to about 25° C.

“Fermentable sugars” refers to a sugar content primarily comprising monosaccharides and some polysaccharides that can be used as a carbon source by a microorganism in a fermentation process to produce a target product.

“Lignocellulosic” refers to material comprising both lignin and cellulose. Lignocellulosic material may also comprise hemicellulose. In the methods described herein, lignin is oxidized and degraded to produce a carbohydrate-enriched biomass comprising fermentable sugars during ozone treatment. Lignin is dissolved and substantially removed from the lignocellulosic biomass during organosolv treatment, both in the absence and presence of ozone, to produce a carbohydrate-enriched biomass comprising fermentable sugars.

“Dissolved lignin” means the lignin that is dissolved in a solvent.

“Al lignin” refers to acid-insoluble lignin.

“Autohydrolysis” refers to the hydrolysis of biomass in the presence of solvent (water or organic solvent plus water) plus heat with no further additions, such as without hydrolytic enzymes

“Cellulosic” refers to a composition comprising cellulose.

“Target product” refers to a chemical, fuel, or chemical building block produced by fermentation. Product is used in a broad sense and includes molecules such as proteins, including, for example, peptides, enzymes, and antibodies. Also contemplated within the definition of target product are ethanol and butanol.

The abbreviation “EtOH” refers to ethanol or ethyl alcohol.

“Dry weight of biomass” refers to the weight of the biomass having all or essentially all water removed. Dry weight is typically measured according to American Society for Testing and Materials (ASTM) Standard E1756-01 (Standard Test Method for Determination of Total Solids in Biomass) or Technical Association of the Pulp and Paper Industry, Inc. (TAPPI) Standard T-412 om-02 (Moisture in Pulp, Paper and Paperboard).

“Selective extraction” means removal of lignin while substantially retaining carbohydrates.

A “solvent solution” and/or “organic solvent solution” as used herein is a solvent mixture in water that includes any organic liquid solvent that dissolves a solid, liquid, or gaseous solute, resulting in a solution. The most suitable solvent solutions for this invention comprise organic solvents such as ethanol, methanol, n-propanol, isopropanol, n-butanol, 2-butanol, isobutanol, t-butanol, pentanol and hexanol and diols with the same number of carbons. They may also include aprotic solvents. The solvent solutions may include additional components in mixture with the solution.

“Biomass” and “lignocellulosic biomass” as used herein refer to any lignocellulosic material, including cellulosic and hemi-cellulosic material, for example, bioenergy crops, agricultural residues, municipal solid waste, industrial solid waste, yard waste, wood, forestry waste, and combinations thereof, and as further described below. Biomass has a carbohydrate content that comprises polysaccharides and oligosaccharides and may also comprise additional components, such as protein and/or lipid.

“Highly conserved” as used herein refers to the carbohydrate content of the lignocellulosic material after the processing steps described herein. In an embodiment of the invention, the highly conserved carbohydrate content provides for sugar yields after saccharification that are substantially similar to theoretical yields and/or demonstration of minimal loss in sugar yield from the processes described herein. In an embodiment of the invention, highly-conserved with reference to carbohydrate content refers to the conservation of greater than or equal to 85% of the biomass carbohydrate as compared to biomass prior to pretreating as described herein.

“Preprocessing” as used herein refers to processing of lignocellulosic biomass prior to pretreatment. Preprocessing is any treatment of biomass that prepares the biomass for pretreatment, such as mechanically chopping and/or drying to the appropriate moisture content.

“Biomass-solvent suspension” refers to a mixture of biomass and solvent wherein the biomass is in suspension in the solvent solution. The biomass-solvent suspension may comprise additional components such as a base or buffer. As used herein, “slurry” is used interchangeably with “suspension.”

“Saccharification” refers to the production of fermentable sugars from polysaccharides by the action of hydrolytic enzymes. Production of fermentable sugars from pretreated biomass occurs by enzymatic saccharification by the action of cellulolytic and hemicellulolytic enzymes.

“Pretreating biomass” or “biomass pretreatment” as used herein refers to subjecting native or preprocessed biomass to chemical, physical, or biological action, or any combination thereof, rendering the biomass more susceptible to enzymatic saccharification or other means of hydrolysis prior to saccharification. For example, the methods claimed herein may be referred to as pretreatment processes that contribute to rendering biomass more accessible to hydrolytic enzymes for saccharification.

“Pretreated biomass” as used herein refers to native or preprocessed biomass that has been subjected to chemical, physical, or biological action, or any combination thereof, rendering the biomass more susceptible to enzymatic saccharification or other means of hydrolysis prior to saccharification.

“Air-drying the filtered biomass” can be performed by allowing the biomass to dry through equilibration with the air of the ambient atmosphere.

“Readily saccharifiable biomass” means biomass that is carbohydrate-enriched and made more amenable to hydrolysis by cellulolytic or hemi-cellulolytic enzymes for producing monomeric and oligomeric sugars.

“Carbohydrate-enriched” as used herein refers to the biomass produced by the process treatments described herein, in which lignin in the biomass is selectively oxidized and degraded (and dissolved and extracted in the case of organosolv) while biomass carbohydrate is retained in good yield. In one embodiment the readily saccharifiable carbohydrate-enriched biomass produced by the processes described herein has a carbohydrate concentration of greater than or equal to about 85% of the biomass carbohydrate as compared to biomass prior to pretreating as described herein while removing 75% or greater of the biomass lignin.

“Heating the biomass suspension” means subjecting the biomass suspended in solvent to a temperature greater than ambient or room temperature.

“Filtering free liquid under pressure” means removal of unbound liquid through filtration, with some pressure difference on opposite faces of the filter.

By “alkaline” is meant a pH of greater than 7.0. Particularly suitable is a pH of the biomass-EtOH/water and an alkaline mixture that is greater than 7.0.

“Air-dried sample” means a pretreated biomass which is allowed to dry at ambient temperature to the point where its moisture content is approximately in equilibrium with that of the ambient air, typically ≧85% dry matter.

“Substantially lignin-free biomass” means a pretreated sample containing about ≦25% of the starting lignin composition.

“Multi-component solvent” means a solvent containing organic solvent, water, and reagents capable of chemical attack on the lignin.

“Pressure vessel” is a sealed vessel that may be equipped or not with a mechanism for agitation of a biomass/solvent suspension, in which a positive pressure is developed upon heating the lignocellulosic biomass.

“Nucleophile” is a chemical reagent capable of forming a covalent bond with its reaction partner by contributing both of the bonding electrons.

“Hydrolysate” refers to the liquid in contact with the lignocellulosic biomass which contains the products of hydrolytic reactions acting upon the biomass (either enzymatic or not), in this case monomeric and oligomeric sugars.

“Organosolv” means a mixture of organic solvent and water.

“Enzyme consortium” or “saccharification enzyme consortium” is a collection of enzymes, usually secreted by a microorganism, which in the present case will typically contain one or more cellulases, xylanases, glycosidases, ligninases and feruloyl esterases.

“Monomeric sugars” or “simple sugars” consist of a single pentose or hexose unit, e.g., glucose.

“Delignification” is the act of removing lignin from lignocellulosic biomass. In the context of this application delignification means 1) fragmentation and extraction of lignin from the lignocellulosic biomass using organosolv under alkaline conditions at elevated temperatures, and/or 2) fragmentation and extraction or degradation of lignin from pretreated lignocellulosic biomass using ozone.

“Fragmentation” is a process in which lignocellulosic biomass is treated under organosolv conditions and/or with ozone to break the lignin down into smaller subunits. In the context of the present application, oxidation of the lignin may contribute to breaking the lignin down into smaller subunits.

“Selective extraction” is a process by which lignin is extracted and dissolved by treatment under organosolv or alkaline organosolv conditions leaving behind the polysaccharide.

“Ozonation” is the act of treating biomass with ozone. The biomass may be present as a biomass-solvent suspension or as a solid without an additional liquid phase.

Methods for pretreating lignocellulosic biomass to produce readily saccharifiable biomass are provided. These methods provide economic processes for rendering components of the lignocellulosic biomass more accessible or more amenable to enzymatic saccharification. The pretreatment may be chemical, physical, or biological, or any combination of the foregoing. In this disclosure the pretreatment method involves first treating biomass under alkaline organosolv conditions, then contacting the treated biomass with a gas comprising ozone. The presence of alkaline organosolv and/or ozone assists lignin fragmentation and removal or degradation and carbohydrate recovery.

In addition, the methods described in the present disclosure minimize the loss of carbohydrate during the pretreatment process and maximize the yield of monomeric sugars in saccharification.

As discussed above the methods described herein include pretreating lignocellulosic material under alkaline organosolv conditions at elevated temperatures, then contacting the treated biomass with a gas comprising ozone to produce a readily saccharifiable carbohydrate-enriched biomass.

### Lignocellulosic Biomass

The lignocellulosic biomass pretreated herein includes, but is not limited to, bioenergy crops, agricultural residues, municipal solid waste, industrial solid waste, sludge from paper manufacture, yard waste, wood and forestry waste. Examples of biomass include, but are not limited to, corn cobs, crop residues such as corn husks, corn stover, grasses, wheat, wheat straw, barley, barley straw, hay, rice straw, switchgrass, waste paper, sugar cane bagasse, sorghum, soy, components obtained from milling of grains, trees, branches, roots, leaves, wood chips, sawdust, shrubs and bushes, vegetables, fruits, flowers and animal manure.

In one embodiment, biomass that is useful for the invention includes biomass that has a relatively high carbohydrate content, is relatively dense, and/or is relatively easy to collect, transport, store and/or handle.

In one embodiment of the invention, biomass that is useful includes corn cobs, corn stover, sugar cane bagasse and switchgrass.

In another embodiment, the lignocellulosic biomass includes agricultural residues such as corn stover, wheat straw, barley straw, oat straw, rice straw, canola straw, and soybean stover; grasses such as switch grass, miscanthus, cord grass, and reed canary grass; fiber process residues such as corn fiber, beet pulp, pulp mill fines and rejects and sugar cane bagasse; sorghum; forestry wastes such as aspen wood, other hardwoods, softwood and sawdust; and post-consumer waste paper products; as well as other crops or sufficiently abundant lignocellulosic material.

The lignocellulosic biomass may be derived from a single source, or biomass may comprise a mixture derived from more than one source; for example, biomass could comprise a mixture of corn cobs and corn stover, or a mixture of stems or stalks and leaves.

The biomass may be used directly as obtained from the source, or may be subjected to some preprocessing, for example, energy may be applied to the biomass to reduce the size, increase the exposed surface area, and/or increase the accessibility of lignin and of cellulose, hemicellulose, and/or oligosaccharides present in the biomass to the alkaline organosolv pretreatment, the ozonation pretreatment, and to saccharification enzymes. Energy means useful for reducing the size, increasing the exposed surface area, and/or increasing the accessibility of the lignin, and the cellulose, hemicellulose, and/or oligosaccharides present in the biomass to the alkaline organosolv pretreatment, the ozonation pretreatment, and to saccharification enzymes include, but are not limited to, milling, crushing, grinding, shredding, chopping, disc refining, ultrasound, and microwave. This application of energy may occur before or during either or both of the pretreatment steps, before or during saccharification, or any combination thereof.

Drying biomass prior to pretreatment may occur as well by conventional means, such as by using rotary dryers, flash dryers, or superheated steam dryers.

### Solvents

The methods described herein include use of an organic solvent solution for pretreating biomass. Solvents useful in the present methods are frequently referred to in the art as Organosolv. Details on pretreatment technologies related to use of solvents and other pretreatments can be found, for example, in Wyman et al., (Bioresource Tech. 96:1959, 2005); Wyman et al., (Bioresource Tech. 96:2026, 2005); Hsu, (“Pretreatment of biomass” In Handbook on Bioethanol: Production and Utilization, Wyman, Taylor and Francis Eds., p. 179-212, 1996); and Mosier et al., (Bioresource Tech. 96:673, 2005). Solvents are used herein for pretreating biomass to remove lignin. Delignification is typically conducted at temperatures of 165-225° C., at liquid to biomass ratios of 4:1 to 20:1, at liquid compositions of 50% organic solvent (volume/volume [v/v]), and for reaction times between 0.5-12 hours. A number of mono- and polyhydroxy-alcohols have been tested as solvents. Ethanol, butanol and phenol have been used (Park, J. K., and Phillips, J. A., Chem. Eng. Comm., 65: 187-205, 1988).

The organosolv or organic solvent pretreatment in the present methods may comprise a mixture of water and an organic solvent at selected condition parameters that include temperature, time, pressure, solvent-to-water ratio and solids-to-liquid ratio. The solvent may comprise, but is not limited to, alcohols, organic acids and ketones. The alcohols may be selected from the group consisting of methanol, ethanol, propanol, isopropanol, butanol, isobutanol, t-butyl alcohol, and mixtures of these. The alcohol may also be a glycol. The concentration of the solvent in solution (i.e. water) in the present invention may be from about 0% to about 100%, or from about 5% to about 95%, or from about 10% to about 90%, or from about 15% to about 85%, or from about 20% to about 80%, or from about 25% to about 75%, or from about 30% to about 70%, or from about 35% to about 65%, or from about 40% to about 60%, or from about 45% to about 55%, or about 50% (v/v). Specifically, for purposes of an embodiment of the methods herein, EtOH/H₂O mixtures from about 50%-90% (v/v) ethanol were examined and found to be effective. In one embodiment, the solvent solution comprises water.

In order to obtain sufficient quantities of sugars from biomass, the biomass may be pretreated under conditions of alkaline organosolv one time or more than one time. To assess performance of the pretreatment and saccharification processes, separately or together, the theoretical yield of sugars derivable from the starting biomass can be determined and compared to measured yields.

### Inorganic Base as an Additional Component of the Solvent Solution

According to the present method, the solvent solution may further comprise at least one inorganic base selected from the group consisting of sodium hydroxide, sodium carbonate, potassium hydroxide, potassium carbonate, calcium hydroxide, calcium carbonate, ammonia, and mixtures thereof as an additional component of the solvent solution. The inorganic base may be added in an amount that is less than about 20 wt % (weight percent) relative to biomass dry weight (w/w biomass). For example, the amount of inorganic base may be about 0.5 wt %, or about 1 wt %, or about 2 wt %, or about 5 wt %, or about 10 wt %, or about 14 wt %, relative to dry weight of biomass. The inorganic base may be used at various concentrations of at least from 0.5 wt % to about 20 wt %. More suitable are the concentrations from about 1 wt % to about 14 wt %. Most suitable are the concentrations between about 5 wt % to about 14 wt %.

In one embodiment, potassium carbonate may be employed as the inorganic base which is an additional component of the solvent solution. Potassium carbonate may be used specifically in an aqueous solution or in an ethanol/water solvent solution. The potassium carbonate may be used at various concentrations, such as, in an amount that is at least about 0.5 wt % to about 20 wt % relative to biomass dry weight (w/w biomass). More suitable are the concentrations from about 1 wt % to about 14 wt %. Most suitable are the concentrations between about 5 wt % to about 14 wt %. Addition of about 5% (w/w biomass) potassium carbonate to a solvent containing about 50%-90% ethanol in water (v/v) may be effective for lignin fragmentation and extraction.

In one embodiment, ammonia may be employed as the inorganic base which may be an additional component of the solvent solution. Ammonia may be used specifically in an aqueous solution or in an ethanol/water solvent solution. The ammonia may be used at various concentrations, such as, in an amount that is at least about 0.5 wt % to about 20 wt % (w/w biomass). More suitable are the concentrations from about 1 wt % to about 14 wt % (w/w biomass). Most suitable are the concentrations between about 5 wt % to about 14 wt % (w/w biomass). Addition of about 8 wt % (w/w biomass) ammonia to a solvent containing about 50%-90% ethanol in water (v/v) may be effective for lignin fragmentation and extraction.

### Organosolv Treatment Conditions

Pretreatment of biomass with the solvent solution comprising at least one inorganic base may be carried out in any suitable vessel. Typically the vessel is one that can withstand pressure, has a mechanism for heating, and has a mechanism for mixing the contents. Commercially available vessels include, for example, the Zipperclave© reactor (Autoclave Engineers, Erie, Pa.), the Jaygo reactor (Jaygo Manufacturing, Inc., Mahwah, N.J.), and a steam gun reactor (described in Geral Methods Autoclave Engineers, Erie, Pa.). Much larger scale reactors with similar capabilities may be used. Alternatively, the biomass and organosolv solution may be combined in one vessel, then transferred to another reactor. Also biomass may be pretreated in one vessel, then further processed in another reactor such as a steam gun reactor.

The pretreatment reaction may be performed in any suitable vessel, such as a batch reactor or a continuous reactor. One skilled in the art will recognize that at higher temperatures (above 100° C.), a pressure vessel is required. The suitable vessel may be equipped with a means, such as impellers, for agitating the biomass-organosolv mixture. Reactor design is discussed in Lin, K.-H., and Van Ness, H. C. (in Perry, R. H. and Chilton, C. H. (eds), Chemical Engineer's Handbook, 5^{th} Edition (1973) Chapter 4, McGraw-Hill, NY). The pretreatment reaction may be carried out as a batch process, or as a continuous process.

Prior to contacting the biomass with solvent solution, vacuum may be applied to the vessel containing the biomass. By evacuating air from the pores of the biomass, better penetration of the solvent solution into the biomass may be achieved. The time period for applying vacuum and the amount of negative pressure that is applied to the biomass will depend on the type of biomass and can be determined empirically so as to achieve optimal pretreatment of the biomass (as measured by the production of fermentable sugars following saccharification).

The heating of the biomass with the solvent solution comprising at least one inorganic base may be carried out at a temperature of from about 100° C. to about 220° C. The heated solution may then be cooled rapidly to room temperature. In another embodiment, the heating of the biomass is carried out at a temperature of about 140° C. to about 180° C. In another embodiment, the heating of the biomass is carried out at a temperature of about 150° C. to about 170° C. Heating of the biomass-solvent suspension may occur for about 15 minutes to about 48 hours, or more preferably from about 1 hour to about 12 hours, or for example from about 1 hour to about 6 hours. In one embodiment, the contacting of the biomass is carried out at a temperature of about 150° C. for about 6 hours.

The contacting of the biomass with the solvent solution comprising at least one inorganic base may be performed at autogeneous pressure. Higher or lower pressures may also be used but are generally less practical.

For the pretreatment process, the temperature, time for pretreatment, solvent solution, inorganic base concentration, biomass concentration, biomass type, and biomass particle size are related; thus these variables may be adjusted as necessary for each type of biomass to optimize the pretreatment processes described herein.

To assess performance of the pretreatment. i.e., the production of readily saccharifiable biomass and subsequent saccharification, separately or together, the theoretical yield of sugars derivable from the starting biomass may be determined and compared to measured yields.

### Ozone

According to the present method, the organosolv-treated biomass, also referred to herein as treated biomass, is contacted with a gas comprising ozone. Ozone treatment promotes oxidation and fragmentation of the lignin and is beneficial to pretreatment, resulting in an increased accessibility of the carbohydrate-enriched biomass to enzymatic saccharification. If a solvent is used during ozone treatment, the solvent may additionally promote extraction of the lignin and/or its oxidized fragments. The use of ozone as a means of lignin removal is relatively selective, leaving the carbohydrates largely intact. In addition, ozone (O₃) easily decomposes to oxygen (O₂) and water, leaving no residue from its use and contributing minimal atmospheric pollution.

The ozone may be generated by any means known in the art, for example from oxygen or air. In the present methods, the gas comprising ozone comprises about 0.1 to about 20 percent by volume ozone, for example about 0.5 to about 5 percent by volume ozone. The gas may further comprise nitrogen, oxygen, argon, or a combination thereof. The gas comprising ozone may also comprise one or more other gases as long as the presence or concentration of the other gases is not deleterious to the ozone treatment. Generally, the ratio of ozone to the pretreated biomass may be at least about 1:1200 on a weight basis, for example for example at least about 1:1000, or at least about 1:750, or at least about 1:500, or at least about 1:200, or at least about 1:100, or at least about 1:50.

### Ozone Treatment Conditions

Contacting of the organosolv-treated biomass with a gas comprising ozone may be carried out in any suitable vessel, such as a batch reactor or a continuous reactor. Typically the vessel is one that has a mechanism for heating or cooling, and has a mechanism for mixing the contents. Optionally, the vessel is one that can withstand pressure. The ozone pretreatment reaction may be performed in a fixed bed reactor, for example, or in a rotating horizontal cylinder, or a continuous stirred tank reactor. The suitable vessel may be equipped with a means, such as impellers, for agitating the biomass or the biomass-solvent suspension, or the vessel itself may rotate or spin to agitate the solid biomass. Reactor design is discussed in Lin, K.-H., and Van Ness, H. C. (in Perry, R. H. and Chilton, C. H. (eds), Chemical Engineer's Handbook, 5^{th} Edition (1973) Chapter 4, McGraw-Hill, NY). The pretreatment reaction may be carried out as a batch process, or as a continuous process. The biomass may be contacted with ozone in the same reactor as the organosolv treatment is performed, or in another reactor. The biomass may be contacted with ozone in one reactor, then saccharified in the same vessel; alternatively, saccharification may be performed in a separate vessel.

Prior to contacting the biomass with a gas comprising ozone, the organosolv-pretreated biomass may be dried by conventional means. The dried organosolv-pretreated biomass may contain about 30 percent to about 70 percent moisture, for example from about 40 percent to about 60 percent moisture.

Contacting the treated biomass with a gas comprising ozone may be carried out at a temperature of from about 0° C. to about 50° C. In one embodiment, the temperature may be from about 0° C. to about 25° C. Higher temperatures may be used but are generally less practical as ozone decomposition increases with increasing temperature. Lower temperatures may also be used but are generally less economical due to cooling requirements and, in the case where a biomass-solvent suspension is used, may not be practical from an operability standpoint. Contacting the treated biomass with a gas comprising ozone may be carried out for a reaction time of at least about 1 minute, for example for a reaction time of about 1 minute to about 60 minutes, or about 1 minute to about 30 minutes, or about 1 minute to about 25 minutes, or about 1 minute to about 20 minutes, or about 1 minute to about 15 minutes, or about 1 minute to about 10 minutes, or about 1 minute to about 5 minutes. Extending the ozonation time beyond that optimal for lignin degradation may result in decreased sugar yields, presumably due to sugar degradation.

Contacting the treated biomass with a gas comprising ozone may be performed at autogeneous pressure. Higher or lower pressures may also be used but are generally less practical.

The treated biomass may be contacted in the solid state with the gas comprising ozone, without a liquid phase being present. Alternatively, the biomass may be contacted as a biomass-solvent suspension with the gas comprising ozone. To generate a biomass-solvent suspension, organosolv-treated biomass may be contacted with water or a mixture of water and an organic solvent (organosolv) as disclosed above. The solvent may comprise, but is not limited to, alcohols, organic acids and ketones. The alcohols may be selected from the group consisting of methanol, ethanol, propanol, isopropanol, butanol, isobutanol, t-butyl alcohol, and mixtures of these. The alcohol may also be a glycol. The concentration of the solvent in solution (i.e. water) in the present invention is from about 0% to about 100%, or from about 5% to about 95%, or from about 10% to about 90%, or from about 15% to about 85%, or from about 20% to about 80%, or from about 25% to about 75%, or from about 30% to about 70%, or from about 35% to about 65%, or from about 40% to about 60%, or from about 45% to about 55%, or about 50% (v/v). Specifically, for purposes of an embodiment of the methods herein, EtOH/H₂O mixtures from about 0%-75% (v/v) ethanol were examined and found to be effective. The solvent used during ozone treatment may be the same or different from the solvent used in the organosolv treatment preceding ozone treatment.

In one embodiment, the solvent may be water. The resulting biomass-solvent suspension may have a pH of about 1 to about 10, for example from about 2 to about 9, or from about 1 to about 7, or from about 1 to about 5. The water may further comprise a buffer, for example a citrate buffer. The selection of an appropriate buffer may be based on the buffer's suitability for controlling pH in a subsequent saccharification. After ozone treatment is complete, the pH of the biomass-solvent suspension may be adjusted to a second pH sufficient for enzymatic saccharification of the biomass, if desired.

For the ozone pretreatment step, the weight percent of biomass in biomass-solvent suspension may be from about 5 weight percent to about 70 weight percent, for example from about 20 weight percent to about 70 weight percent, or from about 30 weight percent to about 60 weight percent.

For the ozone pretreatment step, the temperature, time for pretreatment, ozone concentration in the gas, moisture content, biomass concentration, ratio of ozone to biomass, biomass type, and biomass particle size are related; thus these variables may be adjusted as necessary for each type of biomass to optimize the pretreatment processes described herein.

To assess performance of the pretreatment, i.e., the production of readily saccharifiable biomass, and subsequent saccharification, separately or together, the theoretical yield of sugars derivable from the starting biomass may be determined and compared to measured yields.

### Further Processing

Saccharification

Following pretreatments with organosolv and ozone, the readily saccharifiable biomass comprises a mixture of fragmented lignin and polysaccharides. In the case where ozone treatment is performed on a biomass-solvent suspension, the readily saccharifiable biomass further comprises solvent, extracted lignin, and optionally buffer or an inorganic base. Prior to further processing, the lignin fragments or oxidation products may be removed from the pretreated biomass by filtering and optionally washing the sample with EtOH/H₂O (0% to 100% EtOH volume/volume [v/v]). As the filtration and washing steps are not necessary to obtain improved sugar yields, and as the costs associated with them may negatively impact the economics of the method, filtering and washing of the biomass is preferably omitted in the case where the solvent is water, or where no solvent is used. The biomass may be dried at room temperature, resulting in readily saccharifiable biomass. The concentration of glucan, xylan and acid-insoluble lignin content of the readily saccharifiable biomass may be determined using analytical means well known in the art.

The readily saccharifiable biomass may then be further hydrolyzed in the presence of a saccharification enzyme consortium to release oligosaccharides and/or monosaccharides in a hydrolysate. Surfactants such as polyethylene glycols (PEG) may be added to improve the saccharification process (U.S. Pat. No. 7,354,743 B2, incorporated herein by reference). Saccharification enzymes and methods for biomass treatment are reviewed in Lynd, L. R., et al. (Microbiol. Mol. Biol. Rev., 66:506-577, 2002). The saccharification enzyme consortium may comprise one or more glycosidases; the glycosidases may be selected from the group consisting of cellulose-hydrolyzing glycosidases, hemicellulose-hydrolyzing glycosidases, and starch-hydrolyzing glycosidases. Other enzymes in the saccharification enzyme consortium may include peptidases, lipases, ligninases and feruloyl esterases.

The saccharification enzyme consortium comprises one or more enzymes selected primarily, but not exclusively, from the group “glycosidases” which hydrolyze the ether linkages of di-, oligo-, and polysaccharides and are found in the enzyme classification EC 3.2.1.x (Enzyme Nomenclature 1992, Academic Press, San Diego, Calif. with Supplement 1 (1993), Supplement 2 (1994), Supplement 3 (1995, Supplement 4 (1997) and Supplement 5 [in Eur. J. Biochem., 223:1-5, 1994; Eur. J. Biochem., 232:1-6, 1995; Eur. J. Biochem., 237:1-5, 1996; Eur. J. Biochem., 250:1-6, 1997; and Eur. J. Biochem., 264:610-650 1999, respectively]) of the general group “hydrolases” (EC 3.). Glycosidases useful in the present method can be categorized by the biomass component that they hydrolyze. Glycosidases useful for the present method include cellulose-hydrolyzing glycosidases (for example, cellulases, endoglucanases, exoglucanases, cellobiohydrolases, β-glucosidases), hemicellulose-hydrolyzing glycosidases (for example, xylanases, endoxylanases, exoxylanases, β-xylosidases, arabino-xylanases, mannases, galactases, pectinases, glucuronidases), and starch-hydrolyzing glycosidases (for example, amylases, α-amylases, β-amylases, glucoamylases, α-glucosidases, isoamylases). In addition, it may be useful to add other activities to the saccharification enzyme consortium such as peptidases (EC 3.4.x.y), lipases (EC 3.1.1.x and 3.1.4.x), ligninases (EC 1.11.1.x), and feruloyl esterases (EC 3.1.1.73) to help release polysaccharides from other components of the biomass. It is well known in the art that microorganisms that produce polysaccharide-hydrolyzing enzymes often exhibit an activity, such as cellulose degradation, that is catalyzed by several enzymes or a group of enzymes having different substrate specificities. Thus, a “cellulase” from a microorganism may comprise a group of enzymes, all of which may contribute to the cellulose-degrading activity. Commercial or non-commercial enzyme preparations, such as cellulase, may comprise numerous enzymes depending on the purification scheme utilized to obtain the enzyme. Thus, the saccharification enzyme consortium of the present method may comprise enzyme activity, such as “cellulase”, however it is recognized that this activity may be catalyzed by more than one enzyme.

Saccharification enzymes may be obtained commercially, in isolated form, such as Spezyme® CP cellulase (Genencor International, Rochester, N.Y.) and Multifect® xylanase (Genencor). In addition, saccharification enzymes may be expressed in host organisms at the biofuels plant, including using recombinant microorganisms.

One skilled in the art would know how to determine the effective amount of enzymes to use in the consortium and adjust conditions for optimal enzyme activity. One skilled in the art would also know how to optimize the classes of enzyme activities required within the consortium to obtain optimal saccharification of a given pretreatment product under the selected conditions.

Preferably the saccharification reaction is performed at or near the temperature and pH optima for the saccharification enzymes. The temperature optimum used with the saccharification enzyme consortium in the present method ranges from about 15° C. to about 100° C. In another embodiment, the temperature optimum ranges from about 20° C. to about 80° C. Most typically the temperature optimum ranges from about 45 to about 50° C. The pH optimum may range from about 2 to about 11. In another embodiment, the pH optimum used with the saccharification enzyme consortium in the present method may range from about 4 to about 5.5.

The saccharification may be performed for a time of about several minutes to about 120 hours, and preferably from about several minutes to about 48 hours. The time for the reaction will depend on enzyme concentration and specific activity, as well as the substrate used and the environmental conditions, such as temperature and pH. One skilled in the art can readily determine optimal conditions of temperature, pH and time to be used with a particular substrate and saccharification enzyme(s) consortium.

The saccharification may be performed batch-wise or as a continuous process. The saccharification may also be performed in one step, or in a number of steps. For example, different enzymes required for saccharification may exhibit different pH or temperature optima. A primary treatment may be performed with enzyme(s) at one temperature and pH, followed by secondary or tertiary (or more) treatments with different enzyme(s) at different temperatures and/or pH. In addition, treatment with different enzymes in sequential steps may be at the same pH and/or temperature, or different pHs and temperatures, such as using hemicellulases stable and more active at higher pHs and temperatures followed by cellulases that are active at lower pHs and temperatures.

The degree of solubilization of sugars from biomass following saccharification may be monitored by measuring the release of monosaccharides and oligosaccharides. Methods to measure monosaccharides and oligosaccharides are well known in the art. For example, the concentration of reducing sugars can be determined using the 1,3-dinitrosalicylic (DNS) acid assay (Miller, G. L., Anal. Chem., 31: 426-428, 1959). Alternatively, sugars can be measured by HPLC using an appropriate column as described below.

### Fermentation to Target Products

The readily saccharifiable biomass produced by the present methods may be hydrolyzed by enzymes as described above to produce fermentable sugars which then can be fermented into a target product. “Fermentation” refers to any fermentation process or any process comprising a fermentation step. Target products include, without limitation alcohols (e.g., arabinitol, butanol, ethanol, glycerol, methanol, 1,3-propanediol, sorbitol, and xylitol); organic acids (e.g., acetic acid, acetonic acid, adipic acid, ascorbic acid, citric acid, 2,5-diketo-D-gluconic acid, formic acid, fumaric acid, glucaric acid, gluconic acid, glucuronic acid, glutaric acid, 3-hydroxypropionic acid, itaconic acid, lactic acid, malic acid, malonic acid, oxalic acid, propionic acid, succinic acid, and xylonic acid); ketones (e.g., acetone); amino acids (e.g., aspartic acid, glutamic acid, glycine, lysine, serine, and threonine); gases (e.g., methane, hydrogen (H₂), carbon dioxide (CO₂), and carbon monoxide (CO)).

Fermentation processes also include processes used in the consumable alcohol industry (e.g., beer and wine), dairy industry (e.g., fermented dairy products), leather industry, and tobacco industry.

Further to the above, the sugars produced from saccharifying the pretreated biomass as described herein may be used to produce in general, organic products, chemicals, fuels, commodity and specialty chemicals such as xylose, acetone, acetate, glycine, lysine, organic acids (e.g., lactic acid), 1,3-propanediol, butanediol, glycerol, ethylene glycol, furfural, polyhydroxyalkanoates, cis, cis-muconic acid, and animal feed (Lynd, L. R., Wyman, C. E., and Gerngross, T. U., Biocommodity Engineering, Biotechnol. Prog., 15: 777-793, 1999; and Philippidis, G. P., Cellulose bioconversion technology, in Handbook on Bioethanol: Production and Utilization, Wyman, C. E., ed., Taylor & Francis, Washington, D.C., 179-212, 1996; and Ryu, D. D. Y., and Mandels, M., Cellulases: biosynthesis and applications, Enz. Microb. Technol., 2: 91-102, 1980).

Potential coproducts may also be produced, such as multiple organic products from fermentable carbohydrate. Lignin-rich residues remaining after pretreatment and fermentation can be converted to lignin-derived chemicals, chemical building blocks or used for power production.

Conventional methods of fermentation and/or saccharification are known in the art including, but not limited to, saccharification, fermentation, separate hydrolysis and fermentation (SHF), simultaneous saccharification and fermentation (SSF), simultaneous saccharification and cofermentation (SSCF), hybrid hydrolysis and fermentation (HHF), and direct microbial conversion (DMC).

SHF uses separate process steps to first enzymatically hydrolyze cellulose to sugars such as glucose and xylose and then ferment the sugars to ethanol. In SSF, the enzymatic hydrolysis of cellulose and the fermentation of glucose to ethanol is combined in one step (Philippidis, G. P., in Handbook on Bioethanol: Production and Utilization, Wyman, C. E., ed., Taylor & Francis, Washington, D.C., 179-212, 1996). SSCF includes the cofermentation of multiple sugars (Sheehan, J., and Himmel, M., Bioethanol, Biotechnol. Prog. 15: 817-827, 1999). HHF includes two separate steps carried out in the same reactor but at different temperatures, i.e., high temperature enzymatic saccharification followed by SSF at a lower temperature that the fermentation strain can tolerate. DMC combines all three processes (cellulase production, cellulose hydrolysis, and fermentation) in one step (Lynd, L. R., Weimer, P. J., van Zyl, W. H., and Pretorius, I. S., Microbiol. Mol. Biol. Reviews, 66: 506-577, 2002).

These processes may be used to produce target products from the readily saccharifiable biomass produced by the pretreatment methods described herein.

### Advantages of the Present Methods

One of the advantages of the present methods is the high selectivity for fragmenting and removing lignin from the biomass while leaving the carbohydrates largely intact. Less selective pretreatment methods hydrolyze a portion of the carbohydrates to sugars, for example a portion of the glucans to glucose and/or a portion of the xylans to xylose. If present, the monomeric sugars can be degraded during the pretreatment process, resulting in a decrease in the overall yield to sugar (i.e. through a saccharification step). When aqueous biomass slurries are heated, some of the acetyl groups contained in the biomass may be hydrolyzed to acetic acid, which can lead to carbohydrate (polysaccharide) hydrolysis. The carbohydrate hydrolysis, in turn, can solubilize a portion of the sugars and diminish the overall sugar yields, especially if a biomass washing step follows the pretreatment. In order to prevent acetic acid-induced hydrolysis of carbohydrates, the present method uses an inorganic base. As a result, sugar recoveries are improved and overall sugar recovery (through pretreatment and saccharification steps) is also improved. As demonstrated by the Examples, prolonged ozonation can lead to diminished yields of sugars, in particular xylose. Therefore, there exists an optimal reaction time for ozone treatment, below which the pretreatment will be ineffective, and above which it will be unselective. The optimal reaction time for ozone treatment depends in part on the biomass composition and particle size.

Additionally, lignin is more electron rich than the carbohydrates contained in biomass, and as a result the lignin is more prone to oxidation by the ozone than are the carbohydrates. While not wishing to be bound by any theory, oxidation of the lignin by the ozone is believed to reduce the molecular weight of the lignin fragments, which in turn renders the lignin fragments both more soluble in the solvent solution and less able to bind to cellulolytic enzymes. As a result, the use of lower enzyme loadings in saccharification is enabled, which can provide cost savings with regard to enzyme usage. The present methods advantageously combine treating lignocellulosic biomass with organosolv under alkaline conditions at elevated temperatures followed by selective oxidation of lignin by ozone treatment to produce a readily saccharifiable biomass.

The present methods offer advantageous flexibility regarding ozonation in that the ozone treatment may be performed on solid biomass or on a biomass-solvent suspension. Both options offer opportunity for overall process simplification and economic benefit. For example, if desired the biomass may be treated with ozone as a biomass-solvent suspension, wherein the suspension is formed from an aqueous solution comprising a buffer selected for a subsequent saccharification step. After ozone treatment, the enzyme cocktail may be added directly to the readily saccharifiable biomass and saccharification may be performed in the same reaction vessel. If the solvent solution contains an organic solvent, the readily saccharifiable biomass may be filtered after ozone treatment to further remove lignin from the biomass. Alternatively, solid biomass may be contacted with ozone, that is, without the presence of a liquid phase.

### EXAMPLES

The goal of the experimental work described below was to develop a pretreatment process for lignocellulose that maximized lignin removal and minimized carbohydrate loss in the pretreatment to produce a readily saccharifiable biomass that may be further processed to result in a maximal monomeric sugar yield following enzymatic saccharification. The approach adopted was to selectively extract and fragment the lignin with organosolv treatment under alkaline conditions and at elevated temperatures, then selectively oxidize and fragment the remaining lignin in the presence of a gas comprising ozone, while retaining the sugars with the solids residue. The following experiments show that treatment of lignocellulosic biomass under alkaline organosolv conditions followed by treating with ozone fragmented, extracted, and oxidized the lignin to produce a readily saccharifiable biomass.

The present invention is further defined in the following examples. It should be understood that these examples, while indicating preferred embodiments of the invention, are given by way of illustration only. From the above discussion and these examples, one skilled in the art can ascertain the essential characteristics of this invention, and without departing from the spirit and scope thereof, can make various changes and modifications of the invention to adapt it to various uses and conditions.

The following materials were used in the examples. All commercial reagents were used as received.

Glucose, xylose, cellobiose, and citric acid were obtained from Sigma-Aldrich (St. Louis, Mo.). Ethanol was obtained from Sigma-Aldrich (St. Louis, Mo.). Potassium carbonate was obtained from Sigma-Aldrich (St. Louis, Mo.). The enzyme cocktails were obtained from Genencor (Rochester, N.Y.).

Corn cob was purchased from Independence Corn By Products (ICBP Cob), Independence, Iowa. The seller stored the cob at 60° C. and milled and sieved the cob to ⅛″. The dry mass content of the cob was 95%. Another variety of cob referred to as MD07 cob was obtained from University of Wisconsin Farm, in Madison, Wis. and was hammer milled to ⅜″ particles.

### Carbohydrate Analysis of Biomass

A modified version of the NREL LAP procedure “Determination of Structural Carbohydrates and Lignin in Biomass” was used to determine the weight percent glucan and xylan in the biomass. Sample preparation was simplified by drying at 80° C. under vacuum or at 105° C. under ambient pressure overnight. The samples were knife milled to pass through a 20 mesh screen but were not sieved. The dry milled solids were than subjected to the acid hydrolysis procedure at a 50 mg solids scale. The solids were not first extracted with water or ethanol. HPLC analysis of sugars was done on an Aminex HPX-87H column and no analysis of lignin was attempted.

The soluble sugars glucose, cellobiose, and xylose in saccharification liquor were measured by HPLC (Agilent 1100, Santa Clara, Calif.) using Bio-Rad HPX-87H column (Bio-Rad Laboratories, Hercules, Calif.) with appropriate guard columns, using 0.01 N aqueous sulfuric acid as the eluant. The sample pH was measured and adjusted to 5-6 with sulfuric acid if necessary. The sample was then passed through a 0.2 μm syringe filter directly into an HPLC vial. The HPLC run conditions were as follows:
Biorad Aminex HPX-87H (for carbohydrates):
Injection volume: 10-50 μL, dependent on concentration and detector limits
Mobile phase: 0.01 N aqueous sulfuric acid, 0.2 micron filtered and degassed
Flow rate: 0.6 mL/minute
Column temperature: 50° C., guard column temperature <60° C.
Detector temperature: as close to main column temperature as possible
Detector: refractive index
Run time: 15 minute data collection
   After the run, concentrations in the sample were determined from standard curves for each of the compounds.

Ozone was generated from air using an ozonizer (model CD1500) manufactured by ClearWater Tech (San Luis Obispo, Calif.) and set on maximum voltage. The amount of ozone used in each Example with an ozonation step was calculated from the ozone consumed during the indicated reaction time by measuring the ozone concentration in the ozone-enriched air entering and leaving the experimental apparatus and taking the difference. Ozone measurements were made using a Teledyne Instruments (San Diego, Calif.) ozone monitor, model 450 M.

The following abbreviations are used:

“HPLC” is High Performance Liquid Chromatography, “C” is degrees Centigrade or Celsius; “%” is percent; “mL” is milliliter; “h” is hour(s); “rpm” is revolution per minute; “EtOH” is ethanol; “mg/g” is milligram per gram; “g/100 mL” is gram per 100 milliliter; “g” is gram; “NaOH” is sodium hydroxide; “w/v” is weight per volume; “v/v” is volume for volume, “w/w” is weight for weight; “mm” is millimeter; “mL/min” is milliliter per minute; “min” is minutes; “mM” is millimolar, “N” is normal, “μL” is microliter.

### Comparative Example A

### Control Experiment with Organosolv Only (No Inorganic Base, No Ozone Treatment)

A slurry of ICBP corn cob (240.00 g of 95% dry material) in EtOH (911.0 mL) and water (250.0 mL), was heated to 180° C. for two hours. Upon cooling, the reaction mixture was filtered and washed with 3×250 mL ethanol. The residue was dried in vacuo, at room temperature, to afford 139.1 g residue (61% mass recovery). The residue was then saccharified according to the following procedure.

To a slurry of pretreated corn cob residue from the above procedure (0.499 g) was added 4.093 mL citrate buffer (pH=5), Accellerase® 1000 (46.3 μL, concentration 97.1 mg/mL) and Multifect CX 12 L (26.7 μL, concentration 56.1 mg/mL) enzyme cocktails, and the mixture was left stirring in an incubator/shaker at 48° C. Samples were taken every 24 hours and analyzed by HPLC to determine the monomeric sugar yields versus time. Results are shown in Tables 1 and 2.

### Comparative Example B

### Control Experiment with Organosolv (No Inorganic Base) and Ozone Treatment

Residue obtained from the organosolv treatment of Comparative Example A (1.791 g) was suspended in a mixture of EtOH (10.0 mL) and water (2.69 mL) and a stream of ozone-enriched air (flow rate 2 L/min) was introduced to the slurry for 30 minutes at room temperature; during the reaction time 23 mg of ozone was consumed. The slurry was then filtered to remove the solvent containing dissolved lignin fragments, and the solid was dried in vacuo to generate 1.513 g (84.5%) pretreated biomass, which was saccharified according to the following procedure.

To a slurry of pretreated corn cob residue from the above procedure (0.500 g) was added 3.498 mL citrate buffer (pH=5), Accellerase® 1000 (46.3 μL, concentration 97.1 mg/mL) and Multifect CX 12 L (26.7 μL, concentration 56.1 mg/mL) enzyme cocktails, and the mixture was left stirring in an incubator/shaker at 48° C. Samples were taken every 24 hours and analyzed by HPLC to determine the monomeric sugar yields versus time. Results are shown in Tables 1 and 2.

### Comparative Example C

### Control Experiment with Organosolv (No Inorganic Base) and Ozone Treatment

A slurry of corn cob (15 g of 95% dry material) in EtOH (50.0 mL) was heated with stirring to 180° C. for 12 h. The mixture was then cooled rapidly to room temperature, and filtered. The solid residue was washed with EtOH (50.0 mL), and dried in vacuo to generate 11.25 g (75% mass recovery) of pretreated biomass.

A portion of this material (3.59 g) was suspended in EtOH (16.5 mL) and water (7.28 mL) and a stream of ozone-enriched air (flow rate 3 L/min) was introduced to the slurry for 5 minutes at room temperature; during the reaction time 5 mg of ozone was consumed. The slurry was then filtered to remove the solvent containing dissolved lignin fragments, and the solid was dried in vacuo to generate 3.45 g (99% mass recovery) of pretreated biomass, which was saccharified according to the following procedure.

To a slurry of pretreated biomass from the above procedure (0.499 g) was added citrate buffer (2.995 mL), Accellerase® 1000 (46.3 μL, concentration 97.1 mg/mL) and Multifect CX (26.7 μL, concentration 56.1 mg/mL) enzyme cocktails, and the mixture was left stirring in an incubator/shaker at 48° C. Samples were taken every 24 h and analyzed by HPLC to generate the monomeric sugar yields versus time. The results are shown in Tables 1 and 2.

TABLE 1
Saccharification Yields for Glucose for
Comparative Examples A, B, and C.

| Saccharification | Comparative | Comparative | Comparative |
|---|---|---|---|
| Time (hours) | Example A | Example B | Example C |
| 6 | 8.4 | ND | 34.7 |
| 24 | 34.5 | 34.5 | 42.4 |
| 48 | 44.1 | ND | 49.3 |
| 72 | 49.8 | 58.5 | 52.4 |
| 96 | 59.2 | 68.2 | 59.5 |
| 120 | 62.5 | 69.1 | 62.2 |
| 148 | 65.8 | 78.4 | 73.1 |

Note:
“ND” means Not Determined.

TABLE 2
Saccharification Yields for Xylose for
Comparative Examples A, B, and C.

| Saccharification | Comparative | Comparative | Comparative |
|---|---|---|---|
| Time (hours) | Example A | Example B | Example C |
| 6 | 26.6 | ND | 45.9 |
| 24 | 52.5 | 47.6 | 54.0 |
| 48 | 53.6 | ND | 55.7 |
| 72 | 56.1 | 54.5 | 56.9 |
| 96 | 60.1 | 62.9 | 59.0 |
| 120 | 60.9 | 75.4 | 61.1 |
| 148 | 63.5 | 70.0 | 72.5 |

Note:
“ND” means Not Determined.

### Example 1

### Effect of Alkaline Organosolv and Ozone Treatments

To a slurry of MD07 corn cob (15 g of 91.6% dry material) in EtOH (37.0 mL) and water (15.0 mL) was added potassium carbonate (K₂CO₃) (0.750 g) and the mixture was heated, with stirring, to 180° C. for 12 hours. The mixture was then cooled rapidly to room temperature, and filtered. The solid residue was washed with EtOH (50.0 mL), and dried in vacuo to generate 8.40 g (59% mass recovery) pretreated biomass.

A portion of this material (3.64 g) was suspended in a mixture of EtOH (16.5 mL) and water (7.28 mL) and a stream of ozone-enriched air (flow rate 1.2 L/min) was introduced to the slurry for 30 minutes at room temperature; during the reaction time 8 mg of ozone was consumed. The slurry was then filtered to remove the solvent containing dissolved lignin fragments, and the solid was dried in vacuo to generate 3.175 g (88% mass recovery) of readily saccharifiable biomass, which was saccharified according to the following procedure.

To a slurry of readily saccharifiable biomass from the above procedure (0.499 g) was added citrate buffer (2.995 mL, pH=5), Accellerase® 1000 (46.3 μμL, concentration 97.1 mg/mL) and Multifect CX 12 L (26.7 μL, concentration 56.1 mg/mL) enzyme cocktails, and the mixture was left stirring in an incubator/shaker at 48° C. Samples were taken every 24 hours and analyzed by HPLC to determine the monomeric sugar yields versus time. Results are shown in Tables 3 and 4.

### Example 2

### Effect of Alkaline Organosolv and Ozone Treatments

To a slurry of MD07 corn cob (15 g of 91.6% dry material) in EtOH (50.0 mL) was added K₂CO₃ (0.750 g) and the mixture was heated, with stirring, to 140° C. for 12 hours. The mixture was then cooled rapidly to room temperature, and filtered. The solid residue was washed with EtOH (50.0 mL), and dried in vacuo to generate 12.68 g (88% mass recovery) of pretreated biomass.

A portion of this material (3.60 g) was suspended in a mixture of EtOH (16.5 mL) and water (2.16 mL) and a stream of ozone-enriched air (flow rate 3 L/min) was introduced to the slurry for 30 minutes at room temperature; during the reaction time 15 mg of ozone was consumed. The slurry was then filtered to remove the solvent containing dissolved lignin fragments, and the solid was dried in vacuo to generate 3.499 g (97% mass recovery) of readily saccharifiable biomass, which was saccharified according to the following procedure.

To a slurry of readily saccharifiable biomass from the above procedure (0.503 g) was added citrate buffer (3.018 mL, pH=5), Accellerase® 1000 (46.6 μL, concentration 97.1 mg/mL) and Multifect CX 12 L (26.9 μL, concentration 56.1 mg/mL) enzyme cocktails, and the mixture was left stirring in an incubator/shaker at 48° C. Samples were taken every 24 hours and analyzed by HPLC to generate the monomeric sugar yields versus time. Results are shown in Tables 3 and 4.

### Example 3

### Effect of Alkaline Organosolv and Ozone Treatments

To a slurry of ICBP corn cob (15 g of 95% dry material) in EtOH (37 mL) and water (15 mL) was added K₂CO₃ (0.750 g). The pressure was increased to 200 psi using nitrogen, and the mixture was heated with stirring to 180° C. for 12 h. The mixture was then cooled rapidly to room temperature and filtered. The solid residue was washed with EtOH (50.0 mL), and dried in vacuo to generate 6.60 g (44% mass recovery) of pretreated biomass.

A portion of this material (3.60 g) was suspended in EtOH (16.5 mL) and water (2.16 mL) and a stream of ozone-enriched air (flow rate 3 L/min) was introduced to the slurry for 30 minutes at room temperature; during the reaction time 30 mg of ozone was consumed. The slurry was then filtered to remove the solvent containing dissolved lignin fragments, and the solid was dried in vacuo to generate 3.499 g (97% mass recovery) of readily saccharifiable biomass, which was saccharified according to the following procedure.

To a slurry of readily saccharifiable biomass from the above procedure (0.501 g) was added citrate buffer (3.018 mL), Accellerase® 1000 (46.6 μL, concentration 97.1 mg/mL) and Multifect CX (26.9 μL, concentration 56.1 mg/mL) enzyme cocktails, and the mixture was left stirring in an incubator/shaker at 48° C. Samples were taken every 24 h and analyzed by HPLC to generate the monomeric sugar yields versus time. Results are shown in Tables 3 and 4.

### Example 4

### Effect of Alkaline Organosolv and Ozone Treatments

To a slurry of corn cob (15 g of 95% dry material) in EtOH (50.0 mL) was added K₂CO₃ (0.750 g) and the mixture was heated with stirring to 180° C. for 1 h. The mixture was then cooled rapidly to room temperature and filtered. The solid residue was washed with EtOH (50.0 mL), and dried in vacuo to generate 12.60 g (84% mass recovery) of pretreated biomass.

A portion of this material (3.64 g) was suspended in EtOH (16.5 mL) and water (7.28 mL) and a stream of ozone-enriched air (flow rate 1.2 L/min) was introduced to the slurry for 5 minutes at room temperature; during the reaction time 3 mg of ozone was consumed. The slurry was then filtered to remove the solvent containing dissolved lignin fragments, and the solid was dried in vacuo to generate 3.61 g (99% mass recovery) of readily saccharificable biomass, which was saccharified according to the following procedure.

To a slurry of readily saccharifiable biomass (0.499 g) was added citrate buffer (2.995 mL), Accellerase® 1000 (46.3 μL, concentration 97.1 mg/mL) and Multifect CX (26.7 μL, concentration 56.1 mg/mL) enzyme cocktails, and the mixture was left stirring in an incubator/shaker at 48° C. Samples were taken every 24 h and analyzed by HPLC to generate the monomeric sugar yields versus time. The results are shown in Tables 3 and 4.

TABLE 3
Saccharification Yields for Glucose for Examples 1, 2, 3, and 4.

| Saccharification | | | | |
|---|---|---|---|---|
| Time (hours) | Example 1 | Example 2 | Example 3 | Example 4 |
| 6 | 14.7 | 20.5 | 15.0 | 21.2 |
| 24 | 39.8 | 43.3 | 36.4 | 43.5 |
| 48 | 53.3 | 51.0 | 48.2 | 51.4 |
| 72 | 57.2 | 57.1 | 55.9 | 57.8 |
| 96 | 64.4 | 63.5 | 62.5 | 65.1 |
| 120 | 69.3 | 70.1 | 63.3 | 68.2 |
| 148 | 76.9 | 71.2 | 66.8 | 71.5 |

Note:
“ND” means Not Determined.

TABLE 4
Saccharification Yields for Xylose for Examples 1, 2, 3, and 4.

| Saccharification | | | | |
|---|---|---|---|---|
| Time (hours) | Example 1 | Example 2 | Example 3 | Example 4 |
| 6 | 21.5 | 17.5 | 33.8 | 19.2 |
| 24 | 43.4 | 41.1 | 57.7 | 41.5 |
| 48 | 48.7 | 44.9 | 64.2 | 47.2 |
| 72 | 48.1 | 47.8 | 68.3 | 51.7 |
| 96 | 50.6 | 51.5 | 73.8 | 57.9 |
| 120 | 52.3 | 55.6 | 72.6 | 60.2 |
| 148 | 56.2 | 57.2 | 74.8 | 62.7 |

Note:
“ND” means Not Determined.

The experiments disclosed above were performed in order to uncover the important factors for maximizing monomer sugar yields. It was found that the presence of base in the organosolv treatment step was important for overall sugar recovery, likely due to neutralizing the acid generated from hydrolysis of acetyl groups contained in the lignocellulosic biomass during the organosolv pretreatment. The data also show that ozonolysis is selective in lignin degradation, however prolonged ozone treatment may lead to sugar losses. The data showed that initial pressure during organosolv pretreatment was not important for the outcome of the pretreatment.

Although particular embodiments of the present invention have been described in the foregoing description, it will be understood by those skilled in the art that the invention is capable of numerous modifications, substitutions, and rearrangements without departing from the spirit of essential attributes of the invention. Reference should be made to the appended claims, rather than to the foregoing specification, as indicating the scope of the invention.

What is claimed is:

1. A method for producing readily saccharifiable carbohydrate-enriched biomass, the method comprising:
(a) providing lignocellulosic biomass comprising lignin;
(b) contacting the biomass with an organic solvent solution comprising water and at least one inorganic base selected from the group consisting of sodium hydroxide, sodium carbonate, potassium hydroxide, potassium carbonate, calcium hydroxide, calcium carbonate, ammonia, and mixtures thereof whereby a biomass-solvent suspension is formed;
(c) heating the biomass-solvent suspension to a temperature of about 100° C. to about 220° C. and for a reaction time of about 15 minutes to about 48 hours whereby lignin is fragmented from the biomass and is dissolved in the suspension;
(d) filtering free liquid under pressure after heating the suspension in (c) whereby the dissolved lignin is removed whereby pretreated biomass is formed;
(e) contacting the pretreated biomass from (d) with a gas comprising ozone at a temperature of about 0° C. to about 50° C. and for a reaction time of at least about 1 minute whereby a readily saccharifiable carbohydrate-enriched biomass is produced.

2. The method of claim 1, further comprising:
after (d) and before (e), washing the pretreated biomass produced in step (d) with the organic solvent solution.

3. The method of claim 1, wherein the lignocellulosic biomass is subjected to preprocessing prior to step (a).

4. The method of claim 1, wherein the organic solvent solution further comprises an alcohol selected from the group consisting of methanol, ethanol, n-propanol, isopropanol, n-butanol, 2-butanol, isobutanol, and t-butanol, and mixtures of these.

5. The method of claim 4, wherein the alcohol is ethanol.

6. The method of claim 5, wherein the organic solvent solution contains about 0 to about 100 percent (volume/volume) ethanol.

7. The method of claim 1, wherein the amount of the inorganic base is about 1 weight percent to about 14 weight percent, relative to dry weight of biomass.

8. The method of claim 1, wherein the dry weight of biomass is at a concentration of from about 15% to about 70% of the weight of the biomass-solvent suspension of (b).

9. The method of claim 1, wherein the gas comprises about 0.1 to about 20 percent by volume ozone.

10. The method of claim 1, wherein the gas further comprises air, nitrogen, oxygen, argon, or a combination thereof.

11. The method of claim 1, wherein the ratio of ozone to pretreated biomass in step (e) is at least about 1:1200 on a weight basis.

12. The method of claim 1, further comprising, prior to step (e), a step of contacting the pretreated biomass with a second solvent solution comprising water, whereby a second biomass-solvent suspension is formed.

13. The method of claim 12, wherein the weight percent of biomass in the second biomass-solvent suspension is from about 20 to about 70.

14. The method of claim 1 or 12, wherein the pretreated biomass contains about 30 to about 60 percent moisture.

15. The method of claim 1, further comprising saccharifying the readily saccharifiable carbohydrate-enriched biomass with an enzyme consortium whereby fermentable sugars are produced.

16. The method of claim 15, further comprising fermenting the sugars to produce a target compound.
